(19) United States
(12) Patent Application Publication
(10) Pub. No.: US 2016/0082292 A1
(43) Pub. Date: Mar. 24, 2016

(54) ULTRASONIC TREATMENT DEVICE

(71) Applicant: OLYMPUS CORPORATION, Tokyo (JP)

(72) Inventor: Koichi KUDO, Hachioji-shi (JP)

(73) Assignee: OLYMPUS CORPORATION, Tokyo (JP)

(21) Appl. No.: 14/959,074

(22) Filed: Dec. 4, 2015

Related U.S. Application Data
(63) Continuation of application No. PCT/JP2014/054125, filed on Feb. 21, 2014.

(30) Foreign Application Priority Data
Jun. 4, 2013 (JP) 2013-118023

(51) Int. Cl. A61N 7/00

(52) U.S. Cl. CPC A61N 7/00

(57) ABSTRACT

An ultrasonic treatment device performs a medical treatment by use of an ultrasonic vibration. The ultrasonic treatment device includes a probe, a tubular passage, and a tube path. The ultrasonic vibration is propagated to the probe. The probe includes a treating portion that performs the medical treatment to an object to be treated. The tubular passage is formed along a central axis of the probe and includes a sealing portion at distal position. The tube path is inserted into the tubular passage. The tube path includes a jetting port to jet out a fluid.

### CROSS-REFERENCE TO RELATED APPLICATIONS

This application is a Continuation application of PCT Application No. PCT/JP2014/054125, filed Feb. 21, 2014 and based upon and claiming the benefit of priority from the prior Japanese Patent Application No. 2013-118023, filed Jun. 4, 2013, the entire contents of both of which are incorporated herein by reference.

### BACKGROUND OF THE INVENTION

1. Field of the Invention

This invention relates to an ultrasonic treatment device.

2. Description of the Related Art

An ultrasonic treatment device is known which performs a treatment by use of vibration of an ultrasonic frequency. The ultrasonic treatment device includes a probe that is a treatment tool, and a vibrating member comprising an ultrasonic vibrator. The ultrasonic vibration generated from the ultrasonic vibrator propagates to a tip of the probe.

An operator brings the tip of the probe into contact with a biological tissue or the like which is a treatment object portion of an object to be treated, when performing the treatment. Next, the operator drives the ultrasonic treatment device by operating a switch or the like. At this time, the ultrasonic vibration is given to the biological tissue or the like which comes in contact with the tip of the probe. As a result, frictional heat is generated between the biological tissue or the like and the tip of the probe. This frictional heat and vibration energy by the ultrasonic vibration are utilized, whereby a treatment such as cutting, emulsifying or shattering of the biological tissue or the like is performed.

Usually, in such an ultrasonic treatment device, especially the probe reaches a high temperature due to the frictional heat. When the probe reaches the high temperature, there is the possibility that damage of the biological tissue or the like other than the treatment object portion and breakage of the probe occur. Therefore, the ultrasonic treatment device, especially the probe is preferably maintained at an appropriate temperature.

For example, in an ultrasonic treatment device of a publication of Jpn. Pat. Appln. KOKAI Publication No. 6-38973, a sheath is disposed around a probe to prevent the probe reaching a high temperature. A clearance is interposed between the sheath and the probe. This clearance functions as a flow path through which a fluid to cool the probe having the high temperature flows. Furthermore, the ultrasonic treatment device of the publication of Jpn. Pat. Appln. KOKAI Publication No. 6-38973 has a water supply pump that supplies cooling water to the flow path, and control means for setting a water supply output set value to the water supply pump in accordance with an ultrasonic output set value of an ultrasonic vibrator. At the start of use and during the use of the ultrasonic treatment device of the publication of Jpn. Pat. Appln. KOKAI Publication No. 6-38973, the control means adjusts an amount of water to be supplied by the water supply pump so that the probe maintains its appropriate temperature, thereby cooling the probe. Consequently, in the publication of Jpn. Pat. Appln. KOKAI Publication No. 6-38973, the probe is usable at the appropriate temperature.

### BRIEF SUMMARY OF THE INVENTION

In an ultrasonic treatment device of a publication of Jpn. Pat. Appln. KOKAI Publication No. 6-38973, a water supply tube and a sheath or the like which functions as a water supplying space are disposed around a probe to acquire an amount of water to be supplied to sufficiently cool the probe. As a result, in the ultrasonic treatment device of the publication of Jpn. Pat. Appln. KOKAI Publication No. 6-38973, its size increases in a radial direction that is a vertical direction to a central axis of the probe. This is one factor to disadvantageously increase the size of the ultrasonic treatment device.

Therefore, an object of the present invention is to provide an ultrasonic treatment device which can sufficiently cool a probe and can be miniaturized.

According to an aspect of the invention, there is provided an ultrasonic treatment device which performs a medical treatment by use of an ultrasonic vibration, the device comprising: a probe to which the ultrasonic vibration is propagated and which includes a treating portion that performs the medical treatment to an object to be treated; a tubular passage formed along a central axis of the probe and including a sealing portion at distal position; and a tube path to be inserted into the tubular passage and including a jetting port to jet out a fluid.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute apart of the specification, illustrate embodiments of the invention, and together with the general description given above and the detailed description of the embodiments given below, serve to explain the principles of the invention.

FIG. 1 is a schematic view of an ultrasonic treatment device system;

FIG. 2 is a schematic view of an ultrasonic treatment device;

FIG. 3 is a perspective view of a resonator;

FIG. 4 is a vertical cross-sectional view of a part of a vibrating member;

FIG. 5 is a view showing a cooling mechanism to cool a probe after a treatment;

FIG. 6 is a view showing a cooling mechanism of a second embodiment to cool a piezoelectric element during the treatment;

FIG. 7A is a view showing a cooling mechanism of a third embodiment;

FIG. 7B is a view showing the cooling mechanism of the third embodiment;

FIG. 8 is a vertical cross-sectional view of a part of the cooling mechanism in a state where a valve of a fourth embodiment is opened; and

FIG. 9 is a vertical cross-sectional view of a part of the cooling mechanism in a state where the valve of the fourth embodiment is closed.

### DETAILED DESCRIPTION OF THE INVENTION

Hereinafter, embodiments will be described with reference to the drawings.

### First Embodiment

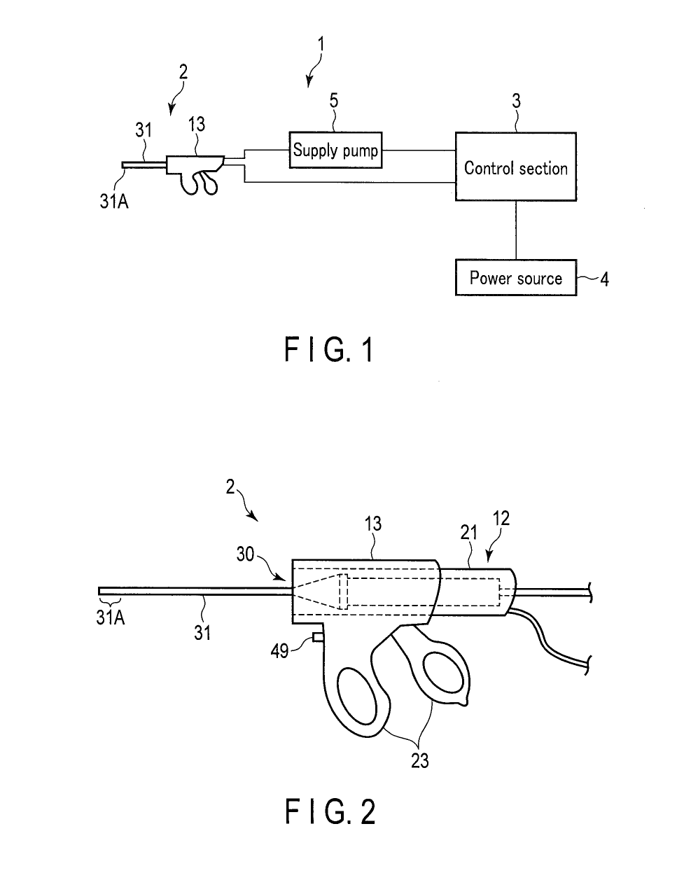

FIG. 1 is a schematic view of an ultrasonic treatment device system 1 of a first embodiment. The ultrasonic treatment device system 1 includes a medical ultrasonic treatment device 2, a control section 3, a power source 4, and a supply pump 5. Although details will be described later, the medical ultrasonic treatment device 2 includes an elongated probe 31, and a handle unit 13 to be held by an operator. When the operator performs a treatment by use of the ultrasonic treatment device 2, the operator holds the handle unit 13 and brings a distal end (a probe distal end) 31A of the probe 31 into contact with a biological tissue or the like (a treatment object portion) of an object to be treated. Hereinafter, a direction in which the probe distal end 31A is disposed will be referred to as a distal side of the ultrasonic treatment device 2, and a direction in which the handle unit 13 is disposed will be referred to as a proximal side of the ultrasonic treatment device 2. The handle unit 13 includes an ultrasonic vibration unit in which a piezoelectric element as an after-mentioned drive source is disposed. The piezoelectric element vibrates (ultrasonically vibrates) at an ultrasonic frequency in one direction, e.g., a vertical direction, when an AC voltage is applied thereto. This vibration propagates to the probe distal end 31A. As a result, the probe distal end 31A vertically vibrates at the ultrasonic frequency. When the vibrating probe distal end 31A comes in contact with the treatment object portion, frictional heat is generated in a contact portion between the probe distal end 31A and the biological tissue or the like. The ultrasonic treatment device 2 performs treatments such as peeling, anastomosis, joining and incision of the treatment object portion by use of vibration energy of the ultrasonic vibration of the probe distal end 31A and the frictional heat generated in the contact portion. When the probe is heated by the frictional heat generated at the probe distal end 31A during the treatment, the probe 31 reaches a high temperature. When the probe 31 has the high temperature even after the treatment ends and when the probe 31 comes in contact with the biological tissue or the like which is not the treatment object portion, there is the possibility that the biological tissue or the like is damaged by heat of the probe 31. Furthermore, due to its high temperature, the probe 31 itself might be broken. Therefore, it is necessary to cool the probe 31 after the treatment is ended.

Therefore, the ultrasonic treatment device 2 has a cooling mechanism to cool the probe 31 heated by the frictional heat. The cooling mechanism includes a flow path to supply a fluid having a temperature at which the probe 31 can be cooled, e.g., a gas such as air or a liquid such as water to the vicinity of the probe distal end 31A. The fluid flowing through this flow path takes heat from the probe 31. In consequence, the probe 31 is cooled. Details of the cooling mechanism will be described later.

The supply pump 5 is connected to the ultrasonic treatment device 2. The supply pump 5 supplies the fluid to the flow path of the ultrasonic treatment device 2. The supply pump 5 is controlled by the control section 3. The control section 3 is connected to the ultrasonic treatment device 2, the supply pump 5 and the power source 4. Power is supplied from the power source 4 to the control section 3. The control section 3 controls driving of the piezoelectric element and the supply pump 5. The control section 3 controls, for example, ON/OFF timing of the driving of the piezoelectric element and amplitude of the vibration of the piezoelectric element. Furthermore, the control section 3 also controls, for example, ON/OFF timing of the driving of the supply pump 5, a flow amount of the fluid and a speed of the fluid.

FIG. 2 is a schematic view of the ultrasonic treatment device 2. The handle unit 13 of the ultrasonic treatment device 2 includes an ultrasonic vibration unit 12. The ultrasonic vibration unit 12 includes a case of an ultrasonically vibrating portion 21 and a resonator 30. The handle unit 13 includes a holding portion 23. The holding portion 23 is a portion to be held by the operator. In the holding portion 23, a switch 49 is disposed.

The switch 49 is disposed in a portion easy to be pressed down when the operator holds the holding portion 23. The portion easy to be pressed down is, for example, a portion at which an index finger of the operator is positioned when the operator holds the holding portion 23. The switch 49 is electrically connected to the control section 3 via a distribution cable or the like (not shown). The switch 49 is, for example, an automatic reset type switch (a momentary switch or a push switch). In this case, when the operator presses down the switch 49, an electric signal concerning an operation of the ultrasonic treatment device 2 is sent to the control section 3. The switch 49 may be disposed at a position other than that of the handle unit 13. In addition, the switch 49 may be, for example, a foot switch.

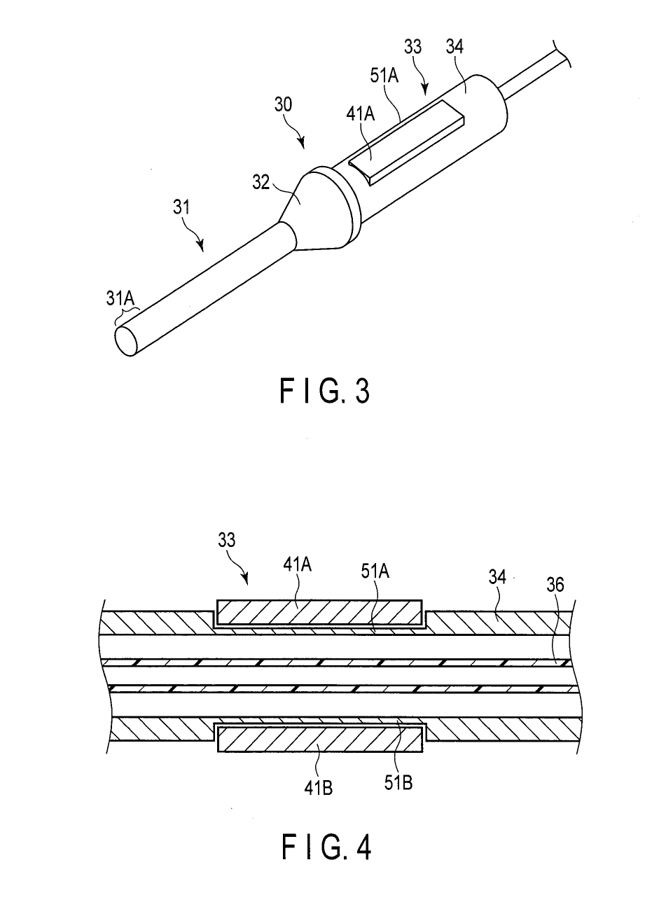

FIG. 3 is a perspective view of the resonator 30. The resonator 30 includes a vibrating member 33, a horn 32, and the probe 31. The vibrating member 33 has, for example, a substantially elongated cylindrical shape. The vibrating member 33 includes an elongated main body 34. The main body 34 is made of, for example, a metal. In the main body 34, there are disposed a first piezoelectric element 41A and a second piezoelectric element 41B (in FIG. 3, only the first piezoelectric element 41A is shown). Hereinafter, the first piezoelectric element 41A and the second piezoelectric element 41B will be referred together as the piezoelectric elements 41A and 41B.

A positional relation between the main body 34 and the piezoelectric elements 41A and 41B will be described with reference to FIG. 4 which is a vertical cross-sectional view of one portion of the vibrating member 33. In an outer periphery of the main body 34, a first groove 51A and a second groove 51B are formed. A shape of a bottom surface of the first groove 51A is, for example, a rectangular shape having a long side parallel to a longitudinal axis of the main body 34. A bottom surface of the second groove 51B also has the same shape as in the first groove 51A. The first groove 51A and the second groove 51B are formed symmetrically to a central axis of the main body 34.

The first piezoelectric element 41A is attached so that its longitudinal axis is parallel to the long side of the bottom surface of the first groove 51A. Similarly, the second piezoelectric element 41B is attached so that its longitudinal axis is parallel to a long side of the bottom surface of the second groove 51B. The first piezoelectric element 41A has, for example, an elongated flat plate shape. The second piezoelectric element 41B has a shape equivalent to that of the first piezoelectric element 41A. Further, the first piezoelectric element 41A is bonded to the first groove 51A to efficiently transmit the vibration thereto. Similarly, the second piezoelectric element 41B is also bonded to the second groove 51B. Further, the piezoelectric elements 41A and 41B are electrically connected to the control section 3 via a distribution cable or the like (not shown). The piezoelectric elements 41A and 41B form a vibration source of the vibrating member 33. The piezoelectric elements 41A and 41B ultrasonically vibrate in a longitudinal direction, when the AC voltage is applied thereto.

On the distal side of the vibrating member 33, the horn 32 is disposed. The horn 32 has a distal end and a proximal end having different shapes. The distal end of the horn 32 has a truncated conical shape whose diameter gradually decreases toward a tip. The proximal end of the horn 32 has a columnar shape slightly extending in the longitudinal direction. On the distal side of the horn 32, the probe 31 is disposed. The probe 31 has, for example, a columnar shape.

As described above, the piezoelectric elements 41A and 41B ultrasonically vibrate when the AC voltage is applied thereto. This ultrasonic vibration propagates to the horn 32 via the main body 34. A lateral sectional area of the horn 32 decreases toward the distal side, and hence the amplitude of the ultrasonic vibration is amplified. The ultrasonic vibration amplified by the horn 32 propagates to the probe 31 and reaches the probe distal end 31A.

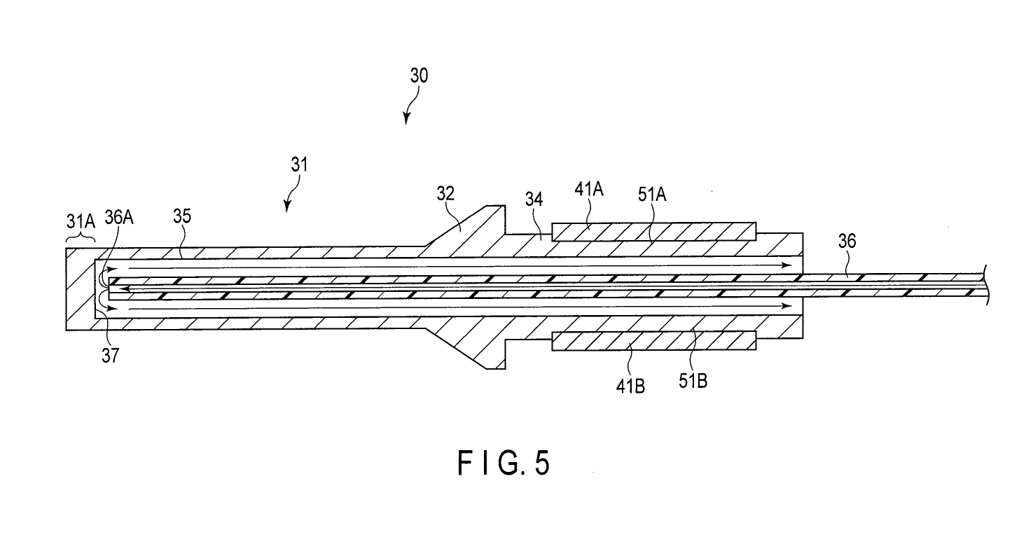

FIG. 5 is a view showing details of the abovementioned cooling mechanism. The cooling mechanism includes a tubular passage 35 and a first tube path 36. The tubular passage 35 is a hole through which the fluid is passed to cool the resonator 30 from the inside. The tubular passage 35 is formed in the resonator 30 to communicate from the proximal end of the vibrating member 33 to the probe distal end 31A along the central axis of the probe 31. The tubular passage 35 has a bottom surface (a sealing portion) 37 in the vicinity of the probe distal end 31A. A lateral cross section of the tubular passage 35 has, for example, a circular shape that is coaxial with the central axis of the probe 31.

The first tube path 36 is, for example, a cylindrical tube. An outer diameter of the first tube path 36 is smaller than that of the tubular passage 35. The first tube path 36 is made of, for example, a material having flexibility. One end of the first tube path 36 is inserted into the tubular passage 35, and the other end thereof is connected to the supply pump 5. In the first tube path 36, the end to be inserted into the tubular passage 35 is referred to as a first tube distal end (a first jetting port) 36A and the end to be connected to the supply pump 5 is referred to as a first tube proximal end. The first jetting port 36A is inserted to the vicinity of the bottom surface 37 that is a portion (a cooling object portion) to be especially prevented from reaching the high temperature so that the port does not come in contact with the bottom surface 37.

The abovementioned flow path includes the tubular passage 35 and the first tube path 36. As shown by arrows in FIG. 5, the fluid is supplied from the supply pump 5, and flows through the first tube path 36 to reach the bottom surface 37. Further, the fluid is inverted on the bottom surface 37, flows along the inside of the tubular passage 35 and the outer periphery of the first tube path 36 toward the proximal end, and is discharged from the proximal end of the resonator 30 to the outside. Hereinafter, a route of the flow of the fluid from the supply pump 5 to the bottom surface 37 will be referred to as an inflow path, and a route of the flow of the fluid from the bottom surface 37 to the proximal end of the resonator 30 will be referred to as an outflow path.

Next, an operation of the present embodiment will be described.

When the treatment is performed, the operator holds the holding portion 23, and attaches the distal end 31A of the probe to the biological tissue that is the treatment object portion. At this time, the operator presses down the switch 49. The control section 3 detects that the switch 49 is pressed down, thereby applying the AC voltage to the piezoelectric elements 41A and 41B. When the AC voltage is applied, the piezoelectric elements 41A and 41B ultrasonically vibrate. This ultrasonic vibration propagates to the probe distal end 31A.

When the probe distal end 31A brought into contact with the treatment object region starts the ultrasonic vibration, frictional heat is generated between the probe distal end 31A and the biological tissue. The operator performs treatments such as the peeling, the anastomosis, the joining and the incision of the treatment object portion by use of the vibration energy of the ultrasonic vibration at the probe distal end 31A and the frictional heat. Due to the frictional heat generated at this time, the probe 31 including the probe distal end 31A reaches the high temperature. When the treatment is ended, the operator releases the switch 49. When the control section 3 detects that the switch 49 is released, the control section stops the applying of the AC voltage to the piezoelectric elements 41A and 41B. Substantially simultaneously, the control section 3 applies a voltage to the supply pump 5 to drive the supply pump 5. When the driving is started, the supply pump 5 supplies the cooling fluid to the inflow path. This fluid flows through the first tube path 36 to jet out toward the bottom surface 37, and takes heat from the probe 31. Further, as shown by the arrows in FIG. 5, the fluid is inverted on the bottom surface 37 to flow in a direction of the outflow path. Further, this fluid flows through the flow path to be discharged from the proximal end of the resonator 30 to the outside of the ultrasonic treatment device 2. In this manner, the supply of the fluid into the flow path is continued for a predetermined time, and hence the probe 31 is sufficiently cooled. After the elapse of the predetermined time, the control section 3 automatically stops the driving of the supply pump 5.

Next, an effect of the present embodiment will be described.

According to the present embodiment, the fluid is passed through the tubular passage 35 formed in the resonator 30 after the treatment, and hence the probe 31 heated by the frictional heat is cooled. As a result, the probe is prevented from reaching the high temperature after the treatment. In consequence, breakage of the probe due to the high temperature is prevented. Furthermore, there is prevented damage of the biological tissue other than the treatment object due to contact with the high-temperature probe. Additionally, in the present embodiment, the first jetting port 36A is disposed in the vicinity of the bottom surface 37. Furthermore, the first jetting port 36A is formed toward the bottom surface 37. According to such a constitution, the fluid is directly jetted out from the first jetting port 36A toward the bottom surface 37. At this time, the fluid directly abuts on the bottom surface 37, and hence there is effectively performed the cooling of the vicinity of the probe distal end 31A which is the portion to be especially cooled.

As compared with a treatment device of a publication of Jpn. Pat. Appln. KOKAI Publication No. 6-38973 in which a cooling flow path is disposed in an outer periphery of a probe, the cooling flow path is formed in the resonator 30 in the ultrasonic treatment device 2 of the present embodiment. Therefore, the ultrasonic treatment device 2 of the present embodiment is miniaturized as compared with the ultrasonic treatment device in which the flow path is present in the outer periphery. Furthermore, the tubular passage 35 is formed to be coaxial with the central axis of the resonator 30. That is, the resonator 30 has an axially symmetric structure. As a result, when the piezoelectric elements 41A and 41B ultrasonically vibrate, an unnecessary vibration mode is not generated in the resonator.

In addition, the control section 3 in the present embodiment appropriately switches the ON/OFF timing of the driving of the piezoelectric elements 41A and 41B and the supply pump 5. In consequence, when the treatment is ended, the cooling of the probe 31 is automatically started by the control section 3, while the operator is not aware. Therefore, during the treatment, temperature rise of the probe distal end 31A that is a treating portion is not obstructed.

At the proximal end of the resonator 30, a suction pump to discharge the fluid may be disposed. This suction pump sucks the fluid flowing through the outflow path from the proximal side of the resonator 30. The ON/OFF timing of the driving of the suction pump is electrically controlled by the control section 3. The probe 31 might be used toward a perpendicular direction. At this time, the fluid is hard to be discharged from the outflow path. When the fluid is a liquid, the fluid is especially hard to be discharged. On the other hand, the fluid is sucked by the suction pump, and hence the fluid flows through the flow path without being stagnated even when the probe 31 is used toward the perpendicular direction. In addition, the fluid is sucked by the suction pump, and hence the fluid discharged from the outflow path is prevented from spilling outward especially when the fluid is the liquid. Furthermore, even when the supply pump 5 has a low output, the fluid is sucked by the suction pump to flow. Therefore, the supply pump 5 can be miniaturized.

In addition, at the proximal end of the tube through which the fluid flows, a pump having functions of both the supply pump 5 and the suction pump may be disposed. This pump is controlled by, for example, the control section 3. For example, the control section 3 drives the pump to jet out the fluid for a predetermined time. Next, the control section 3 drives the pump to suck out the fluid gathered in the tubular passage 35. In this case, it is not necessary to divide the inflow path and the outflow path.

Furthermore, the vibrating member 33 of the present embodiment has the piezoelectric elements 41A and 41B each having the flat plate shape. The vibrating member 33 may be of a Langevin structure.

Furthermore, the cooling mechanism may include a temperature sensor. When the switch 49 is released to end the treatment, the temperature sensor senses the temperature of a predetermined position of the probe 31. When this sensed temperature is in excess of a predetermined temperature, the control section 3 turns ON the driving of the supply pump 5. Further, when the temperature of the predetermined position is the predetermined temperature or less, the control section 3 turns OFF the driving of the supply pump 5. According to such a constitution, the cooling is performed at more appropriate timing.

Furthermore, the switches 49 may be disposed to individually control driving operations of the piezoelectric elements 41A and 41B and the supply pump 5. For example, two switches are disposed. In this case, for example, one switch is a switch to switch an ON/OFF state of the driving of the ultrasonic treatment device 2, and the other switch is a switch to switch an ON/OFF state of the driving of the supply pump 5.

Furthermore, a jaw may be disposed in the vicinity of the probe distal end 31A. At this time, the holding portion 23 is a movable handle. When this handle is moved, the jaw also moves. The jaw is disposed in the vicinity of the probe distal end 31A, and hence when the treatment is performed, the operator can firmly sandwich the biological tissue that is the treatment object region by the jaw.

### Second Embodiment

A second embodiment will be described with reference to the drawings. It is to be noted that in the second embodiment, detailed descriptions of a constitution equivalent to the first embodiment are omitted.

When piezoelectric elements 41A and 41B are ultrasonically vibrated by applying an AC voltage thereto, a part of vibration energy and electric energy of the piezoelectric elements 41A and 41B is converted into heat. Therefore, the piezoelectric elements 41A and 41B generate heat to reach a high temperature. When the piezoelectric elements 41A and 41B reach the high temperature, there is the possibility that a fluctuation of a resonance frequency and breakage of the piezoelectric elements 41A and 41B are caused. Therefore, during a treatment, the piezoelectric elements 41A and 41B are preferably cooled.

In an ultrasonic treatment device 2 of the present embodiment, a first tube path 36 is moved at appropriate timing to cool each of a probe 31 and the piezoelectric elements 41A and 41B. Therefore, in the present embodiment, the first tube path 36 has, for example, a moving mechanism (not shown) on a first tube proximal end side. The moving mechanism includes, for example, a linear motor, a stepping motor, and an SMA actuator. The moving mechanism is connected to a control section 3, and driving of the moving mechanism is controlled by the control section 3. The moving mechanism moves the first tube path 36 in a longitudinal axis direction. More specifically, the moving mechanism moves the first tube path 36 in, for example, a direction to insert the first tube path into a tubular passage 35 and a direction to extract the first tube path from the tubular passage 35. The moving mechanism moves the first tube path 36 in a longitudinal direction, whereby a position of a first jetting port 36A in the tubular passage 35 is adjusted. In consequence, the first jetting port 36A can be moved to the vicinity of a probe distal end 31A or a distal end of each of the piezoelectric elements 41A and 41B.

The control section 3 electrically controls timing of driving of the moving mechanism, a driving direction and a driving amount. For example, when an operator presses down a switch 49, the control section 3 sends an electric signal to the moving mechanism so that the driving turns ON. The moving mechanism that has received this electric signal moves the first tube path 36 in a longitudinal direction.

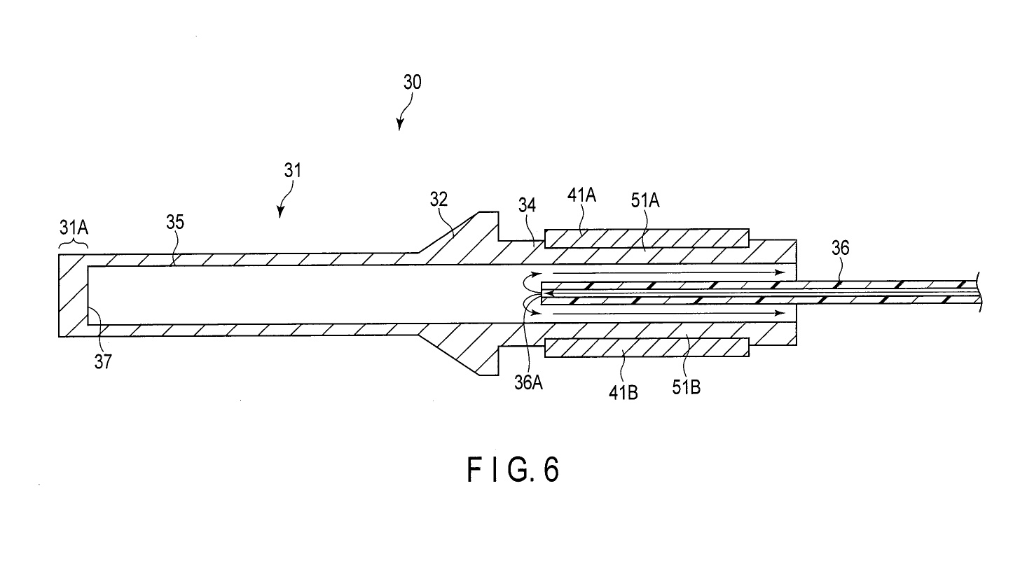

When performing a treatment, the operator presses down the switch 49. The control section 3 detects that the switch 49 is pressed down, thereby turning ON the driving of the piezoelectric elements 41A and 41B. Simultaneously, the control section 3 drives the moving mechanism to position the first jetting port 36A in the vicinity of the distal ends of the piezoelectric elements 41A and 41B. When the first jetting port 36A is positioned in the vicinity of the distal ends of the piezoelectric elements 41A and 41B, the control section 3 turns ON driving of a supply pump 5. When the driving of the supply pump 5 turns ON, a fluid jets out from the first jetting port 36A. This fluid flows from the first jetting port 36A toward a proximal side by convection as shown by arrows in FIG. 6. At this time, the fluid takes heat from the piezoelectric elements 41A and 41B, and is discharged from the proximal end of a resonator 30 to the outside of the ultrasonic treatment device 2.

When the treatment is ended, the operator releases the switch 49. The control section 3 detects that the switch 49 is released, thereby turning OFF the driving of the piezoelectric elements 41A and 41B. At this time, the control section 3 maintains a state where the supply pump 5 is driven. Furthermore, the control section 3 drives the moving mechanism to position the first jetting port 36A in the vicinity of a bottom surface 37. As shown by arrows in FIG. 5, the fluid jetting from the first jetting port 36A is inverted on the bottom surface 37 to flow toward an outflow path, and flows through the outflow path to be discharged from the proximal end of the resonator 30 to the outside of the ultrasonic treatment device 2. At this time, the fluid takes heat from the probe 31, especially the probe distal end 31A. Thus, supply of the fluid into a flow path is continued for a predetermined time, and hence the probe 31 is sufficiently cooled. After the elapse of the predetermined time, the control section 3 automatically stops the driving of the supply pump 5.

According to the present embodiment, by the moving mechanism, the first jetting port 36A is disposed in the vicinity of the distal ends of the piezoelectric elements 41A and 41B during the treatment, and disposed in the vicinity of the probe distal end 31A after the treatment. Therefore, both of the bottom surface 37 and the piezoelectric element 41A and 41B can be cooled at appropriate timing. In addition to the probe 31, the piezoelectric elements 41A and 41B can be cooled, and hence it is possible to prevent a fluctuation of a resonance frequency and damage in a case where the piezoelectric elements 41A and 41B reach the high temperature.

Here, in an ultrasonic treatment device which does not cool the piezoelectric elements 41A and 41B, a device is required which tracks a resonance frequency so that resonance occurs even in a case where the resonance frequency fluctuates due to a temperature change of the piezoelectric elements 41A and 41B. On the other hand, in the ultrasonic treatment device 2 of the present embodiment, the piezoelectric elements 41A and 41B are cooled, and hence the resonance frequency of the piezoelectric elements 41A and 41B hardly fluctuates before the driving and after the driving. In consequence, the ultrasonic treatment device 2 of the present embodiment does not require the resonance frequency tracking device. Therefore, the ultrasonic treatment device 2 of the present embodiment is miniaturized.

In addition, when the operator only presses down the switch 49, the control section 3 drives the moving mechanism at the appropriate timing and the driving amount. At this time, the moving mechanism disposes the first jetting port 36A at an appropriate position. Thus, the moving mechanism is controlled by the control section 3, and hence the first jetting port 36A is disposed at the appropriate position during the treatment and after the treatment while the operator is not aware.

### Third Embodiment

A third embodiment will be described with reference to the drawings. It is to be noted that in the third embodiment, detailed descriptions of a constitution equivalent to the first embodiment are omitted.

In the present embodiment, to cool a probe 31 and piezoelectric elements 41A and 41B, a plurality of (two) jetting ports are disposed so that fluids are jetted out to the probe 31 and the piezoelectric elements 41A and 41B, respectively.

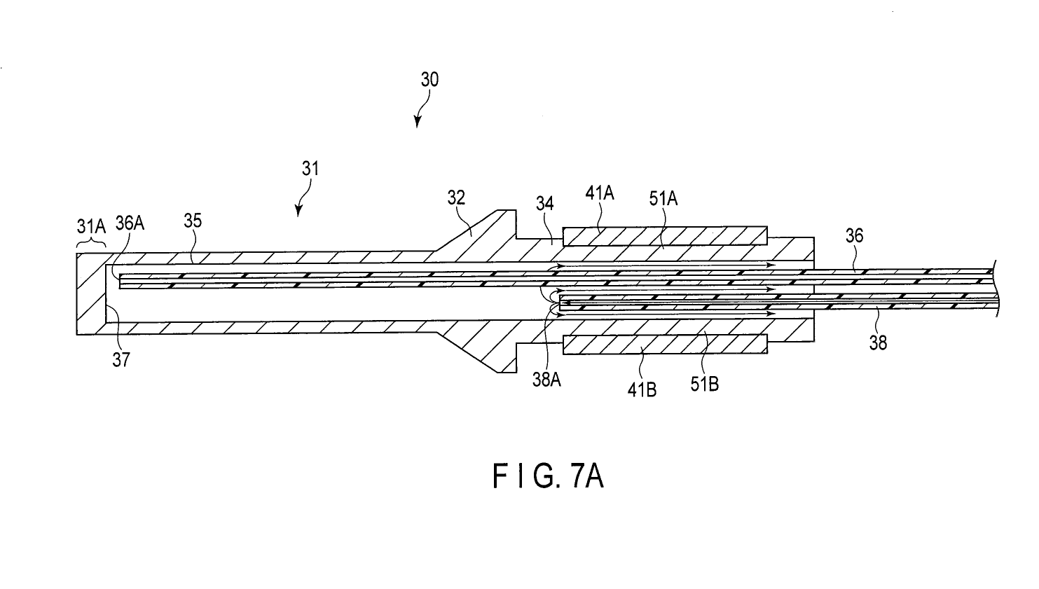

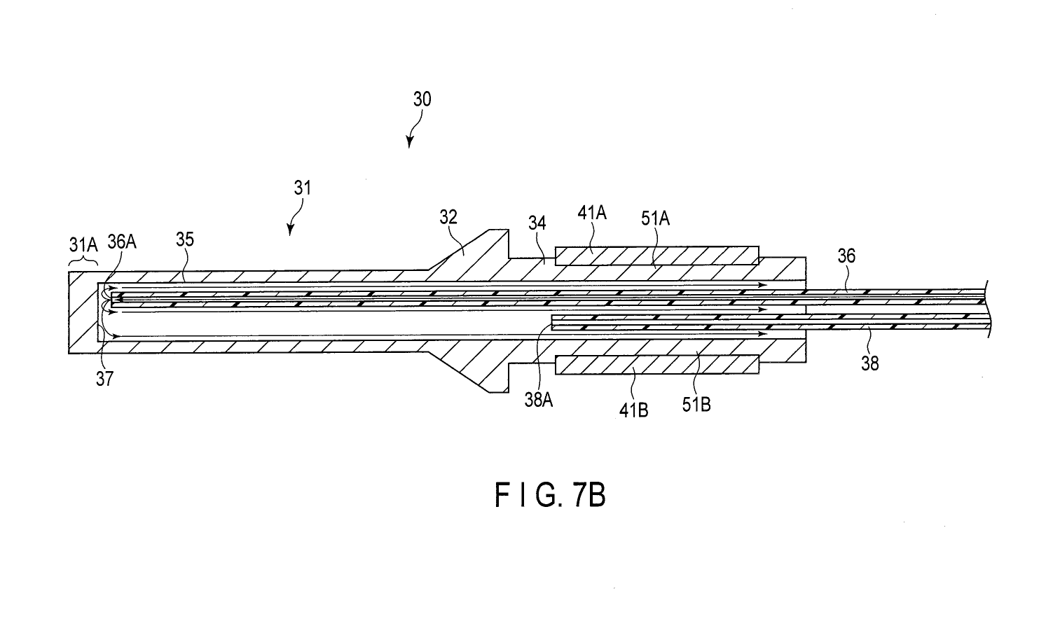

FIG. 7A and FIG. 7B are views showing a cooling mechanism of the present embodiment. The cooling mechanism includes a first tube path 36 and a second tube path 38. The second tube path 38 is, for example, a cylindrical tube similar to the first tube path 36. The second tube path 38 is made of, for example, a material having flexibility. One end of the second tube path 38 is inserted together with the first tube path 36 into a tubular passage 35, and the other end thereof is connected to a supply pump 5. Similarly to the first tube path 36, also in a second tube path 38, one end inserted into the tubular passage 35 is referred to as a second tube distal end (a second jetting port) 38A, and the other end connected to the supply pump 5 is referred to as a second tube proximal end. The second jetting port 38A is disposed in the vicinity of distal ends of the piezoelectric elements 41A and 41B in the tubular passage 35. A first jetting port 36A is disposed in the vicinity of a probe distal end 31A in the tubular passage 35.

The supply pump 5 also supplies a fluid to the second tube path 38 in addition to the first tube path 36. A control section 3 electrically controls the supply pump 5 to supply the fluid to the first tube path 36 and the second tube path 38 at appropriate timing. Specifically, the control section 3 controls the supply pump 5 to supply the fluid only to the second tube path 38 during a treatment. Furthermore, the control section 3 controls the supply pump 5 to supply the fluid only to the first tube path 36 after the treatment.

When the treatment is performed, an operator presses down a switch 49. The control section 3 detects that the switch 49 is pressed down, thereby turning ON driving of the piezoelectric elements 41A and 41B and the supply pump 5. At this time, the control section 3 controls the supply pump 5 so that the fluid is not supplied to the first tube path 36 and is supplied only to the second tube path 38. As shown in arrows in FIG. 7A, the fluid supplied to the second tube path 38 is jetted out from the second jetting port 38A, inverted in a direction of an outflow path by convection, and discharged from a proximal end to the outside of an ultrasonic treatment device 2. At this time, the fluid takes heat from the piezoelectric elements 41A and 41B.

When the treatment is ended, the operator releases the switch 49. The control section 3 detects that the switch 49 is released, thereby turning OFF the driving of the piezoelectric elements 41A and 41B. At this time, the control section 3 controls the supply pump 5 so that the fluid is supplied only to the first tube path 36 and is not supplied to the second tube path 38. As shown in FIG. 7B, the fluid supplied to the first tube path 36 is jetted out from the first jetting port 36A, abuts on a bottom surface 37 to be inverted, and flows through an outflow path to be discharged from a proximal end of a resonator 30 to the outside of the ultrasonic treatment device 2. At this time, the fluid takes heat from the probe 31, especially the probe distal end 31A. Thus, the supply of the fluid into a flow path is continued for predetermined time, and hence the probe 31 is sufficiently cooled. After elapse of the predetermined time, the control section 3 automatically stops driving of the supply pump 5.

According to the present embodiment, the second jetting port 38A is disposed at distal ends of the piezoelectric elements 41A and 41B in the tubular passage 35, and the first jetting port 36A is disposed at the probe distal end 31A. The ultrasonic treatment device 2 of the present embodiment does not require a moving mechanism to move a tube. Therefore, the control section 3 can appropriately cool the piezoelectric elements 41A and 41B and the probe distal end 31A only by controlling switching of a position from which the fluid jets out as compared with the second embodiment.

It is to be noted that in the present embodiment, as tubes through which the fluid flows, two tubes, i.e., the first tube path 36 and the second tube path 38 are disposed, but three or more tubes may be disposed.

### Fourth Embodiment

A fourth embodiment will be described with reference to the drawings. It is to be noted that in the fourth embodiment, detailed descriptions of a constitution equivalent to the first embodiment are omitted.

In the present embodiment, a hole is formed in a part of an outer periphery of a first tube path 36. In this hole, a movable valve 61 is disposed to appropriately switch a position from which a fluid jets out during a treatment and after the treatment.

The first tube path 36 of the present embodiment includes a hole (a third jetting port) 36B in a part of the outer periphery. In addition, the first tube path 36 includes the openable/closable valve 61 to close the third jetting port 36B. The third jetting port 36B is opened toward a radial direction that is a direction vertical to a longitudinal axis. The third jetting port 36B is disposed in the vicinity of distal ends of piezoelectric elements 41A and 41B in a tubular passage 35.

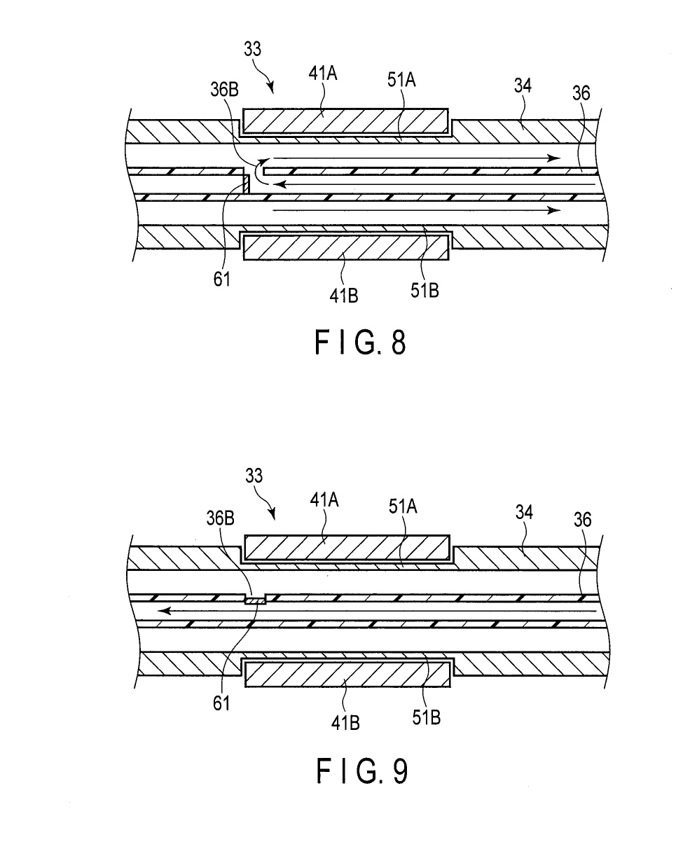

The valve 61 is movably fixed to, for example, the inside of the first tube path 36. The opening/closing of the valve 61 is controlled by a control section 3. The valve 61 as in, for example, an electronic valve is electrically controlled by the control section 3. The opening/closing of the valve 61 may be controlled by, for example, a mechanical mechanism. In addition, the opening/closing of the valve 61 may be controlled by, for example, a pressure of the fluid. The control section 3 receives an electric signal to open and close the valve 61. During the treatment, as shown in FIG. 8, the control section 3 opens the valve 61 to close a first jetting port 36A and to open the third jetting port 36B. Additionally, as shown in FIG. 9, after the treatment, the control section 3 closes the valve 61 to open the first jetting port 36A and to close the third jetting port 36B.

When the treatment is performed, an operator presses down a switch 49. The control section 3 detects that the switch 49 is pressed down, thereby turning ON driving of the piezoelectric elements 41A and 41B and a supply pump 5. The control section 3 simultaneously opens the valve 61. At this time, an inflow path is opened to communicate with the third jetting port 36B. In consequence, the fluid jets out from the third jetting port 36B. As shown by arrows in FIG. 8, this fluid jets out from the third jetting port 36B, is inverted in a direction of an outflow path by convection, and is discharged from a proximal end to the outside of an ultrasonic treatment device 2. At this time, the fluid takes heat from the piezoelectric elements 41A and 41B.

When the treatment is ended, the operator releases the switch 49. The control section 3 detects that the switch 49 is released, thereby turning OFF the driving of the piezoelectric elements 41A and 41B and maintaining a state where the supply pump 5 is driven. The control section 3 simultaneously closes the valve 61. At this time, the inflow path is opened to communicate with the first jetting port 36A. In consequence, the fluid jets out from the first jetting port 36A. As shown by an arrow in FIG. 9, this fluid jets out from the first jetting port 36A toward a bottom surface 37, is inverted on the bottom surface 37, and flows through the outflow path to be discharged from a proximal end of a resonator 30 to the outside of the ultrasonic treatment device 2. At this time, the fluid takes heat from a probe 31, especially a probe distal end 31A. Thus, supply of the fluid into a flow path is continued for predetermined time, and hence the probe 31 is sufficiently cooled. After the elapse of the predetermined time, the control section 3 automatically stops the driving of the supply pump 5.

According to the present embodiment, the opening/closing of the valve 61 is only controlled to cool the piezoelectric elements 41A and 41B during the treatment and to cool the probe 31 after the treatment, so that a position from which the fluid jets out can be switched. Therefore, as compared with the above respective embodiments, it is not necessary to dispose a moving mechanism and two or more tubes. Further, only by opening and closing the valve 61 disposed in the first tube path 36, the piezoelectric elements 41A and 41B and the probe 31, especially the probe distal end 31A can be cooled at appropriate timing.

What is claimed is:

1. An ultrasonic treatment device which performs a medical treatment by use of an ultrasonic vibration, the device comprising:
a probe to which the ultrasonic vibration is propagated and which includes a treating portion that performs the medical treatment to an object to be treated;
a tubular passage formed along a central axis of the probe and including a sealing portion at distal position; and
a tube path to be inserted into the tubular passage and including a jetting port to jet out a fluid.

2. The ultrasonic treatment device according to claim 1,
wherein the jetting port of the tube path is disposed at a position from which the fluid jets out to a heated portion of the probe.

3. The ultrasonic treatment device according to claim 1, comprising:
a vibrating member that performs the ultrasonic vibration; and
a horn that amplifies the ultrasonic vibration and in which the vibrating member is disposed on a proximal side and the probe is disposed on a distal side,
wherein the tubular passage communicates from a proximal end of the vibrating member to a distal end of the probe.

4. The ultrasonic treatment device according to claim 3,
wherein the fluid is jetted out to a heated portion of the vibrating member during the medical treatment, and
the fluid is jetted out to a heated portion of the treating portion after the medical treatment.

5. The ultrasonic treatment device according to claim 1,
wherein a diameter of the tube path is smaller than a diameter of the tubular passage, and
a flow path is formed so that the fluid flows through a hollow portion of the tube path and a clearance between the tubular passage and an outer surface of the tube path.

6. The ultrasonic treatment device according to claim 4, further comprising:
a moving mechanism that moves the tube path,
wherein the moving mechanism moves the jetting port to a position from which the fluid jets out to a heated portion of the piezoelectric element during the medical treatment and moves the jetting port to a position from which the fluid jets out to a heated portion of the treating portion after the medical treatment.

7. The ultrasonic treatment device according to claim 4,
wherein the tube paths are inserted into the tubular passage, and
the jetting ports of the tube paths are disposed at a position from which the fluid jets out to a heated portion of the vibrating member and a position from which the fluid jets out to a heated portion of the treating portion, respectively.

8. The ultrasonic treatment device according to claim 4, which includes:
a hole formed in the tube path; and
a valve that selectively closes the hole, wherein the valve closes the hole to jet out the fluid from the hole to a heated portion of the vibrating member during the medical treatment, and to jet out the fluid from the jetting port to a heated portion of the treating portion after the medical treatment.
